(19) United States
(12) Patent Application Publication
(10) Pub. No.: US 2008/0221647 A1
(43) Pub. Date: Sep. 11, 2008

(54) SYSTEM AND METHOD FOR MONITORING PHOTODYNAMIC THERAPY

(75) Inventors: David L. Chamberland, Medford, OR (US); Xueding Wang, Canton, MI (US); Paul Carson, Ann Arbor, MI (US); David Wood, Birmingham, MI (US); James Montie, Ann Arbor, MI (US)

Correspondence Address: BROOKS KUSHMAN P.C., 1000 TOWN CENTER, TWENTY-SECOND FLOOR, SOUTHFIELD, MI, 48075, US

(73) Assignee: THE REGENTS OF THE UNIVERSITY OF MICHIGAN, Ann Arbor, MI (US)

(21) Appl. No.: 12/036,677

(22) Filed: Feb. 25, 2008

Related U.S. Application Data
(60) Provisional application No. 60/891,283, filed on Feb. 23, 2007.

(51) Int. Cl. A61N 5/06; G06K 9/20

(52) U.S. Cl. 607/88; 382/131

(57) ABSTRACT

A system and method for monitoring photodynamic therapy of a target tissue, where the target tissue contains a photosensitizing substance, include a first light source configured to deliver light to the target tissue, the first light source having a wavelength capable of exciting the photosensitizing substance. An ultrasonic transducer receives photoacoustic signals generated due to optical absorption of light energy by the target tissue, and a control unit in communication with the ultrasonic transducer reconstructs photoacoustic tomographic images from the received photoacoustic signals to provide an indication of optical energy deposition due to the photosensitizing substance in the target tissue.

### CROSS-REFERENCE TO RELATED APPLICATIONS

This application claims the benefit of U.S. provisional application Ser. No. 60/891,283 filed Feb. 23, 2007, which is incorporated by reference herein.

### BACKGROUND OF THE INVENTION

1. Field of the Invention

This invention relates to a system and method for monitoring photodynamic therapy.

2. Background Art

Photodynamic therapy (PDT) represents a relatively new approach to the treatment of various cancers and nonmalignant, hyper-proliferative diseases. Approved by the FDA, PDT is presently being used for esophageal cancer and early stage lung cancer. It is also being utilized as an investigational therapy for obstructive lung cancer, Barrett's esophagus, head and neck, and prostate cancer. PDT is particularly suited to use in head and neck cancers and prostate cancer because of its ability to minimize damage to nerves and blood vessels adjacent to the tumor, and to preserve functions of organs.

PDT relies on photo excitation of an inactive photosensitizing drug in the target organ, tissue, or cells of interest at a wavelength matched to photosensitizer absorption. The excited photosensitizer reacts in situ with molecular oxygen to produce cytotoxic reactive oxygen species, resulting in necrosis of the treated target. PDT-associated photo-consumption of oxygen and hemodynamic insults that include capillary occlusion, hemorrhage, and stasis are important for the development of necrosis and target eradication. PDT therefore requires oxygen to cause target damage. However, therapy itself can deplete target oxygenation, thereby self-limiting its power. The effect of PDT on target oxygenation is highly dependent on choice of photosensitizer, drug-light interval, and fluence rate. Accordingly, in vivo monitoring of target oxygen levels, or possibly other substances, before, during, and after PDT treatment has great clinical significance.

### BRIEF DESCRIPTION OF THE DRAWING

FIG. 1 is a schematic diagram of a system for monitoring photodynamic therapy according to an aspect of the present invention.

### DETAILED DESCRIPTION OF THE INVENTION

As required, detailed embodiments of the present invention are disclosed herein; however, it is to be understood that the disclosed embodiments are merely exemplary of the invention that may be embodied in various and alternative forms. The figures are not necessarily to scale, some features may be exaggerated or minimized to show details of particular components. Therefore, specific structural and functional details disclosed herein are not to be interpreted as limiting, but merely as a representative basis for teaching one skilled in the art to variously employ the present invention.

The present invention includes a system and method which may be used for the monitoring, guidance, and evaluation of photodynamic therapy (PDT) using photoacoustic technology or any multimodality system utilizing photoacoustic technology. During PDT, a photosensitizing substance is applied in a target tissue. Photoacoustic technology according to the present invention is able to describe the distribution of optical energy deposition in tissues due to not only the intrinsic optical absorption, but also the optical absorption brought by the photosensitizing substance (or any other substance including, but not limited to, a pharmaceutical substance, biologic substance, or optical contrast agent). As a result, the system and method according to the present invention are able to describe the spatial distribution and dynamic change of the photosensitizing substance in target tissues along with biological structures and functional hemodynamic properties (e.g., blood oxygen saturation).

Photoacoustic imaging and sensing technology employs optical signals to generate ultrasonic waves, and may be utilized for imaging tissue structures and functional changes, and describing the optical energy deposition in biological tissues with both high spatial resolution and high sensitivity. For example, in photoacoustic tomography (PAT), a short-pulsed electromagnetic source—such as a tunable pulsed laser source, pulsed radio frequency (RF) source or pulsed lamp—is used to irradiate a biological sample. The photoacoustic (ultrasonic) waves excited by thermoelastic expansion are then measured by highly sensitive detection device, such as ultrasonic transducer(s) made from piezoelectric materials and optical transducer(s) based on interferometry. Photoacoustic images are reconstructed from detected photoacoustic signals generated due to optical absorption in the sample through a reconstruction algorithm, where the intensity of photoacoustic signals is proportional to optical energy deposition.

Optical signals, employed in PAT to generate ultrasonic waves in biological tissues, present high electromagnetic contrast between various tissues and also enable highly sensitive detection and monitoring of tissue abnormalities. It has been shown that optical imaging is much more sensitive to detect early stage cancers than ultrasound imaging and X-ray computed tomography. The optical signals can present the molecular conformation of biological tissue and are related to significant physiologic parameters, such as tissue oxygenation and hemoglobin concentration. Traditional optical imaging modalities suffer from low spatial resolution in imaging subsurface biological tissues due to the overwhelming scattering of light in tissues. In contrast, the spatial resolution of PAT is only diffraction-limited by the detected photoacoustic waves rather than by optical diffusion; consequently, the resolution of PAT is excellent (60 microns, adjustable with the bandwidth of detected photoacoustic signals). Besides the combination of high electromagnetic contrast and high ultrasonic resolution, the advantages of PAT also include good imaging depth, enabling imaging of anatomical areas such as a finger joint as a whole organ, gathering of spectroscopic information of molecular components and biochemical changes, relatively low cost, non-invasive, non-ionizing, and compatible with current ultrasonography systems to enable multi-modality imaging.

Functional spectroscopic photoacoustic tomography (SPAT) is able to study the spectroscopic absorption properties in biological tissues with high sensitivity, high specificity, good spatial resolution and good imaging depth. In SPAT, laser pulses at two or more wavelengths are applied to the biological sample sequentially. Then, high resolution photoacoustic images of the sample at each wavelength can be obtained. With the measured photoacoustic images as a function of wavelength, local spectroscopic absorption in the sample can be studied, which presents both morphological and functional information. This technology enables the spectral identification and mapping of a biological and biochemical substance in the localized areas in the specimen, including, but not limited to, hemoglobin, lipid, water, and cytochromes. The volumetrically distributed spectroscopic information can be used for noninvasive, serial in vivo identification purposes of different intrinsic biological tissues in the setting of disease diagnosis, disease progression, and monitoring of tissue changes during treatments, not limited to drug therapies. Besides intrinsic contrast in biological tissues, SPAT can also visualize and quantify the dynamic distribution of extrinsic optical contrast agents in living tissues including, but not limited to, biological dyes and gold nanoparticles.

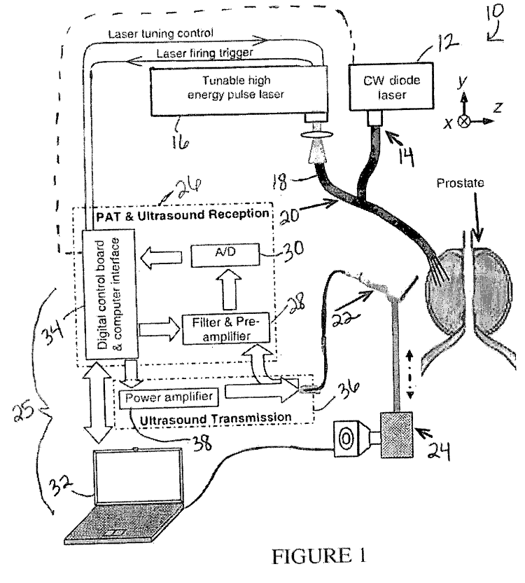

A PAT-guided PDT therapeutic system according to an aspect of the present invention is shown in FIG. 1 and designated generally by reference numeral 10, wherein such a configuration may be used, for example, for monitoring the treatment of prostate cancer. System 10 may include at least one light source or laser 12 for producing light energy in the form of light pulses or continuous waves which can be delivered to the local or distant target tissue, such as through a catheter via optical fibers 14, a fluid core light guide, or the like. In one embodiment, the target tissue may include the prostate. Of course, any catheter and target tissue location is fully contemplated in accordance with the present invention. Furthermore, it is understood that “target tissue” as used herein may refer to any area of a living organism or non-living media.

PDT relies on photo excitation of an inactive photosensitizing drug in the target organ, tissue, or cells of interest at a wavelength matched to photosensitizer absorption. According to one aspect of the present invention, the wavelength of light source 12 is selected to excite the photosensitizing drug, such that the drug may react in situ with molecular oxygen to produce cytotoxic reactive oxygen species, thereby resulting in necrosis of the treated target tissue, such as the prostate. In one embodiment, a continuous wave (CW) light or a laser with long pulse duration (e.g., on the order of microseconds) may be utilized by light source 12 for therapeutic purposes. In one non-limiting example, light source 12 for PDT may be provided by a diode laser (e.g. 732-nm diode laser; Diomed), but could be any wavelength laser. For PDT purposes, light source 12 may be any device that can provide CW or pulsed light, such as, but not limited to, a diode laser, dye lasers, and arc lamps.

If light source 12 used for therapy is a pulsed laser with short pulse duration, this light source 12 may also enable photoacoustic imaging. In particular, when pulsed light is absorbed by the target tissue, photoacoustic waves will be generated due to the optical absorption of biological tissues (i.e., optical energy deposition). Therefore, light source 12 may generate laser pulses utilized for both therapeutic and PAT purposes, wherein the light provided by light source 12 may have a tunable wavelength.

Since CW or long pulse duration light may not generate high quality photoacoustic images, a separate PAT laser source can be employed according to the present invention. As shown in FIG. 1, a second light source 16, such as a high energy pulse laser (e.g., Ti:Sapphire laser, optical parametric oscillator (OPO) system, dye laser, and arc lamp), may be provided to deliver light pulses to the target tissue. For example, an OPO (Vibrant B, Opotek) pumped by an Nd:YAG laser (Brilliant B, Bigsky) may provide laser pulses in the NIR region. In general, light source 16 may provide pulses with a duration on the order of nanoseconds (e.g., 5 ns) and a narrow linewidth (e.g., on the order of nanometers) for irradiating the target tissue. The wavelength of light source 16 may be tunable over a broad region (e.g., from 300 nm to 1850 nm), but is not limited to any specific range. The selection of the laser spectrum region depends on the imaging purpose, specifically the biochemical substances to be studied. In general, the light source used for SPAT according to the present invention may be any device that can provide short light pulses with high energy, short linewidth, and tunable wavelength, and other configurations are also fully contemplated. Light source 16 may be connected to an optical fiber bundle 18 or the like which may deliver laser light to the target tissue via coupling of fiber bundles 14, 18 into a Y-shaped optical coupler 20 or other means, such that the light from light sources 12 and 16 may be delivered to the same location in the target tissue, such as the prostate.

The photoacoustic signals can be scanned by a diagnostic ultrasound platform, such as in a transrectal manner, to reconstruct photoacoustic images as described below. When light source 16 is operating at the same wavelength as light source 12 for PDT, or when a single light source is used for both PDT and PAT, a structural photoacoustic image may be obtained which presents the distribution of light dose, the optical absorption, and the effective attenuation coefficient in the tissue under the PDT treatment. For example, the foci and borders of target tissue may be identified. To image hemodynamic parameters in the target tissue, SPAT may be performed at other wavelengths (e.g., 800 nm) than the wavelength for PDT (e.g., 732 nm). Imaging at two or more wavelengths enables an absolute estimation of blood oxygenation and a relative estimation of blood volume in the tissue under the PDT treatment at any time (e.g., before, during, or after treatment), which may permit interactive adjustment of treatment intensity. Again, because the light for SPAT (e.g., from light source 16) and the light for PDT (e.g., from light source 12) may be delivered to exactly the same locations in the tissue, photoacoustic imaging provides a direct and essentially real time monitoring and evaluation of the PDT effect. According to another aspect of the present invention, laser pulses at wavelengths for sensing and enabling image and spectroscopic data acquisition can be interspersed with therapeutic laser pulses, whether from a single light source or separate light sources.

As indicated above, the photoacoustic signals may be detected external to the human body by a transducer 22, such as a high-sensitivity, wide-bandwidth ultrasonic transducer, and used to reconstruct photoacoustic images using PAT. Transducer 22 can be any ultrasound detection device, e.g. single element transducers, 1D or 2D transducer arrays, optical transducers, transducers of commercial ultrasound machines, and others. The photoacoustic signals can be scanned along any surfaces around the target tissue. Moreover, detection at the detection points may occur at any suitable time relative to each other.

More particularly, the parameters of ultrasonic transducer 22 include element shape, element number, array geometry, array central frequency, detection bandwidth, sensitivity, and others. Transducers with designs such as, but not limited to, linear, arcuate, circular, and 2D arrays, can be applied for photoacoustic signal receiving, wherein the design of transducer 22 may be determined by the shape and location of the studied tissue, the expected spatial resolution and sensitivity, the imaging depth, and others. In general, transducer 22 may include a 1D array that is able to achieve 2D imaging of the cross section in the tissue with single laser pulse. The imaging of a 3D volume in the tissue can be realized by scanning the array along its axis (e.g., along y-axis in FIG. 1), such as with a computer-controlled translation stage 24. In order to achieve 3D photoacoustic imaging at one wavelength with a single laser pulse, a 2D transducer array could instead be employed for signal detection.

Besides the extra-vascular ultrasound detection described herein, the photoacoustic signals generated by laser pulses according to the present invention may also be measured through an intravascular or endoscopic ultrasound technique. In this case, a small ultrasonic transducer (not shown) could be inserted into a vessel, orifice, or any body cavity through a catheter together with an optical fiber (or light guide). The ultrasonic transducer may be positioned very close to the site of the target tissue and may scan the light-generated photoacoustic signals for imaging and sensing.

The received photoacoustic signals may be processed by a control unit 25 comprising reception circuitry 26, optionally including a filter and pre-amplifier 28 and an A/D converter 30, and a computer 32 in communication with a digital control board and computer interface 34. Digital control board and computer interface 34 may also receive the triggers from light source 16. At the same time, control unit 25 may also control the tuning of the wavelength of light source 16 through digital control board and computer interface 34. A “computer” may refer to any suitable device operable to execute instructions and manipulate data, for example, a personal computer, work station, network computer, personal digital assistant, one or more microprocessors within these or other devices, or any other suitable processing device. It is understood that reception circuitry 26 shown in FIG. 1 is only an example, and that other circuitries with similar functions may also be employed in system 10 according to the present invention for control and signal receiving.

The detected photoacoustic signals can be processed by computer 32 and utilized for 3D image reconstruction utilizing PAT. Photoacoustic tomographic images presenting the tissue structures and abnormalities and a map of the optical energy deposition of the target tissue may be generated with both high spatial and temporal resolution through any basic or advanced reconstruction algorithms based on diffusing theory, back-projection, filtered back-projection, and others. The reconstruction of optical images may be performed in both the spatial domain and frequency domain. PAT produces a real time image and overlying energy map for the operating physician to guide the amount of applied energy focused on the target tissue while preserving surrounding tissue. Therefore, with the system and method of the present invention, the physician may be provided with a real time evaluation of tissue responses to therapy, such that the treatment plan may be adjusted on-line. Before or after the generation of photoacoustic, optical and ultrasound images, any signal processing methods can be applied to improve the imaging quality. Photoacoustic images may be displayed on computer 32 or another display.

As described above, pulsed light from light source 16 (or light source 12 if it is properly configured) can induce photoacoustic signals in the target tissue that are detected by ultrasonic transducer 22 to generate 2D or 3D photoacoustic tomographic images of the target tissue (e.g., prostate) and surrounding tissues. By varying the light wavelength in the tunable region and applying laser pulses at two or more wavelengths to the tissue, the local spectroscopic absorption of each point in the target tissue can be generated and analyzed using computer 32. The photoacoustic image presents the optical absorption distribution in biological tissues, while spectroscopic photoacoustic data reveal not only the morphological information but also functional biochemical information in biological tissues. Spectroscopic photoacoustic tomography (SPAT) may yield high resolution images and point-by-point spectral curves for substance identification within a three-dimensional specimen, such as biological organs.

At each voxel in a three dimensional area, a spectroscopic curve indicating the concentration of various absorbing materials can be produced. The subsequent mapped point-by-point spectroscopic curves of the obtained tissue image can describe spatially distributed biological and biochemical substances including, but not limited to, intrinsic biological parameters such as glucose, hemoglobin, cytochromes, blood concentration, water concentration, and lipid concentration along with functional parameters such as oxygen saturation. Extrinsic entities including, but not limited to, molecular or cellular probes, markers, antibodies, or pharmaceutical or contrast agents added for any therapeutic or diagnostic reason, including image enhancement or refined molecular or cellular mapping, could also be incorporated in the system and method described herein. The system and method according to the present invention could also be used for point to point treatment, i.e. once a characteristic spectral curve is detected at any three-dimensional location within the target tissue, thermal or photoacoustic signals could be directed to that location for therapies needing photoactivation of a pharmaceutical compound, such as in PDT.

Referring again to FIG. 1, by using ultrasonic transducer 22 as both a transmitter and receiver of signals, ultrasound signal transmission may also be achieved through an ultrasound transmission system 36 in communication with digital control board and computer interface 34. Ultrasound transmission system 36 is capable of generating high voltage pulses and corresponding delays for each transducer element, and may include an amplifier 38. A pulse-echo technique may be used for pure ultrasound imaging. The whole transducer array or overlapping sub arrays can be used to transmit and receive ultrasound pulses and then generate ultrasound images of the target tissue through the technique of synthetic aperture. Multiple transmissions can be used for each subarray position in order to create multiple focal zones and thereby achieve uniform illumination along the propagation path. System 10 according to the present invention can realize not only gray scale ultrasound images to present tissue morphology in 2D or 3D space, but also Doppler ultrasound images to depict real-time blood flow in biological tissues and provide another assessment of the therapeutic effect. The photoacoustic and ultrasound imaging results of the same target tissue may be combined together through image registration and used to provide very comprehensive diagnostic information.

In accordance with the present invention, the PAT and ultrasound reception and the ultrasound transmission in FIG. 1 can be realized with any proper design of circuitry 26, 36. The circuitry performs as an interface between the computer 32 and transducer 22, light source 16, and other devices. “Interface” may refer to any suitable structure of a device operable to receive signal input, send control output, perform suitable processing of the input or output or both, or any combination of the preceding, and may comprise one or more ports, conversion software, or both. A component of a reception system may comprise any suitable interface, logic, processor, memory, or any combination of the preceding.

According to another aspect of the present invention, control unit 25 may function to control light source 12. Through such an integrated control unit, both control and monitoring of the therapeutic procedure may be achieved. The integrated control unit may generate and analyze point-by-point imaging and spectroscopic information of tissues under therapy. Through programming, this control unit may shut off the laser light automatically through a feedback system.

PDT-associated photo-consumption of oxygen and hemodynamic insults that include capillary occlusion, hemorrhage, and stasis are important for the development of necrosis and target eradication. PDT therefore requires oxygen to cause target damage, but therapy itself can deplete target oxygenation, thereby self-limiting its power. The effect of PDT on target oxygenation is highly dependent on choice of photosensitizer, drug-light interval, and fluence rate. Using the system and method described herein, in vivo monitoring of target tissue oxygen levels before, during, and after PDT treatment may be accomplished.

Several relationships regarding optical energy deposition applicable to the system and method according to the present invention will now be described. If the electromagnetic pumping pulse duration is much shorter than the thermal diffusion time, thermal diffusion can be neglected; this is known as the assumption of thermal confinement. In this case, the acoustic wave p(r,t) is related to electromagnetic absorption H(r,t) by the following wave equation:

$$\frac{1}{{c}^{2}} \frac{{\partial }^{2} p ( r , t )}{\partial {t}^{2}} - {∇}^{2} p ( r , t ) = \frac{β}{{C}_{p}} \frac{\partial H ( r , t )}{\partial t} , \qquad ( 1 )$$

where c is the acoustic velocity, Cₚ is the specific heat, and β is the coefficient of volume thermal expansion. The solution based on Green's function can be expressed as:

$$p ( r , t ) = \frac{β}{4 π {C}_{p}} \int \int \int \frac{{d}^{3} {r}^{′}}{\vert r - {r}^{′} \vert} \frac{\partial H ( {r}^{′} , {t}^{′} )}{\partial {t}^{′}} {\vert}_{{t}^{′} = t - ( \vert r - {r}^{′} \vert / c )} , \qquad ( 2 )$$

The source term H(r,t) can further be written as the product of a purely spatial and a purely temporal component i.e.,

H(r,t)=A(r)I(t). (3)

Substituting Eq. (3) into Eq. (2) results in

$$p ( r , t ) = \frac{β}{4 π {C}_{p}} \int \int \int \frac{{d}^{3} {r}^{′}}{\vert r - {r}^{′} \vert} A ( {r}^{′} ) \frac{d I ( {t}^{′} )}{d {t}^{′}} . \qquad ( 4 )$$

The function A(r) is the spatially distributed optical energy deposition that can be written as

A(r)=φ(r)μₐ(r), (5)

where φ(r) is the distribution of light fluence and μₐ(r) is the distribution of optical absorption. When the temporal profile I(t) of the heating pulse is a Dirac delta function, Eq. 4 can be written as

$$p ( r , t ) = \frac{β}{4 π {C}_{p}} \int \int \int \frac{{d}^{3} {r}^{′}}{\vert r - {r}^{′} \vert} A ( {r}^{′} ) {δ}^{′} ( t - \frac{\vert {r}_{0} - r \vert}{c} ) . \qquad ( 6 )$$

And we have

$$p ( r , t ) = \frac{β {c}^{2}}{4 π {C}_{p}} \frac{\partial }{\partial t} [ t {\int \int }_{R = ct} A ( {r}^{′} ) d s ] , \qquad ( 7 )$$

which is an integration to be carried out on the surface of a sphere with a radius of R=ct around the observation point.

One problem with PAT may involve reconstructing the distribution of optical energy deposition A(r) from the collected photoacoustic signals. Assuming that the photoacoustic measurement is realized along a spherical surface around the target tissue and the detection radius r₀ is much larger than the wavelengths of the photoacoustic waves that are useful for imaging, the photoacoustic image can be reconstructed with the following equation:

$$A ( r ) = - \frac{{r}_{0}^{2} {C}_{p}}{2 π β {c}^{4}} \int {\int }_{s} d s \frac{1}{t} \frac{\partial p ( {r}_{0} , t )}{\partial t} {\vert}_{t = \vert {r}_{0} - r \vert / c} , \qquad ( 8 )$$

which is an integration carried along the scanning surface S.

Again, the image of A(r) obtained by PAT presents the optical energy deposition in the target tissue which is a product of the light fluence φ(r) (i.e., light dose) and the tissue optical absorption coefficient μₐ(r). When μₐ(r) in the PDT treatment area are nearly homogeneous (μₐ(r)−μₐ), which is a reasonable assumption considering the limited penetration of light in biological tissues, photoacoustic images may describe the distribution of light fluence φ(r) delivered by the illumination of optical fibers.

Besides the light dose distribution, the intensity and the shape of photoacoustic images enable measurements of local tissue optical properties, including the absorption coefficient μₐ and the effective attenuation coefficient μ_{eff} surrounding the illumination fibers. μ_{eff} can be expressed as μ_{eff}=\sqrt{3μₐ(μ′ₛ+μₐ)}, where μ′ₛ is the reduced scattering coefficient. With the diffusion approximation, the light fluence rate φ(r) at a distance r from a point source can be expressed as

$$φ ( r ) = \frac{{I}_{0} {μ}_{eff}^{2}}{4 π {μ}_{a}} \frac{{e}^{- {μ}_{eff} r}}{r} , \qquad ( 9 )$$

where I₀ is the source strength. The relative distribution of the light fluence φ(r), or in other words the attenuation of light fluence as a function of the distance r from the point source, is determined by μ_{eff} only. A photoacoustic image presents the spatially distributed φ(r) at different locations in tissues around each illumination fiber, which can be used to evaluate the tissue effective attenuation coefficient μ_{eff}. In theory, measurements of φ(r) at two different distances r from the output end of an illumination fiber are sufficient to determine μ_{eff}. Photoacoustic images provide the measurements at multiple sites, enable more accurate evaluation of μ_{eff}, and allow evaluation of the variation of μ_{eff} within the treatment area.

At the output end of an illumination fiber, assuming the refractive index in tissues is consistent, the light fluence rate will be independent of the location in the target tissue. Therefore, the photoacoustic measurement (e.g., optical energy deposition) at the output end of an illumination fiber is proportional to the local optical absorption coefficient μₐ in the tissue. After a calibration by using a phantom with known optical properties, the photoacoustic imaging system 10 will be able to quantify the optical absorption coefficient μₐ of tissues around the illumination fibers for PDT. As such, the system and method according to the present invention may describe light energy distribution and therefore permit interactive adjustment of the direction and intensity of the light beam during therapy.

In SPAT, photoacoustic imaging may be performed at two or more optical wavelengths. Then, the absorption coefficients of the biological tissue under the PDT treatment can be measured at two or more wavelengths. Similar to NIRS, SPAT relies on the spectroscopic differences between the two types of hemoglobin, oxygenated hemoglobin (HbO₂) and deoxygenated hemoglobin (Hb). When HbO₂ and Hb are dominant absorbing chromophores in a biological sample (which is the case herein), the measured absorption coefficients of the sample at two wavelengths (μₐ^{λ}^{₁} and μₐ^{λ}^{₂}) can be used to compute the concentrations of these two forms of hemoglobin. Further, the functional hemodynamic parameters, including hemoglobin oxygen saturation (SO₂; blood oxygenation) and total hemoglobin concentration (HbT; blood volume), can be computed in the tissue under the PDT treatment by solving the following two equations:

$$\begin{matrix}{SO}_{2} = \frac{[ Hb {O}_{2} ]}{[ Hb {O}_{2} ] + [ Hb ]} = \frac{{μ}_{a}^{{λ}_{2}} {ɛ}_{Hb}^{{λ}_{1}} - {μ}_{a}^{{λ}_{1}} {ɛ}_{Hb}^{{λ}_{2}}}{{μ}_{a}^{{λ}_{1}} {ɛ}_{Δ Hb}^{{λ}_{2}} - {μ}_{a}^{{λ}_{2}} {ɛ}_{Δ Hb}^{{λ}_{1}}} , & ( 10 ) \\ Hb T = [ Hb {O}_{2} ] + [ Hb ] = \frac{{μ}_{a}^{{λ}_{1}} {ɛ}_{Δ Hb}^{{λ}_{2}} - {μ}_{a}^{{λ}_{2}} {ɛ}_{Hb}^{{λ}_{1}}}{{ɛ}_{Hb}^{{λ}_{1}} {ɛ}_{Hb {O}_{2}}^{{λ}_{2}} - {ɛ}_{Hb}^{{λ}_{2}} {ɛ}_{Hb {O}_{2}}^{{λ}_{1}}} . & ( 11 )\end{matrix}$$

where ε_{HbO}_{₂} and ε_{Hb} are the molar extinction coefficients of HbO₂ and Hb, respectively; ε_{ΔHb}=ε_{HbO}_{₂}−ε_{Hb}; and [HbO₂] and [Hb] are the concentrations of HbO₂ and Hb, respectively. This measurement based on SPAT enables an absolute estimation of blood oxygenation and a relative estimation of blood volume (blood flow) in the local tissue under the PDT treatment.

Several techniques have been explored previously for measuring tissue oxygenation and its correlated blood flow and blood oxygenation during the course of PDT. BOLD-contrast magnetic resonance imaging (MRI) is curbed by its high cost and poor imaging unit mobility, limiting its use for real-time applications. Laser Doppler and optical coherence tomography (OCT) typically measure only the tissue surface (penetration depth<1 mm). Near-infrared spectroscopy (NIRS) has limited spatial resolution (worse than 1 cm in most cases) due to the overwhelming scattering of light in biological tissues. Power Doppler ultrasound does not readily permit continuous measurement during PDT. Moreover, ultrasound technology is not able to measure tissue blood oxygenation and blood volume.

The PAT system 10 according to the present invention includes high soft tissue contrast, high accuracy in describing light dose distribution, high sensitivity to hemodynamic changes, good spatial resolution, and sufficient imaging depth, which may greatly benefit the evaluation and optimization of PDT of cancer and other disorders. Because PAT is able to differentiate malignant from benign tissues, it may guide the positioning of illumination fibers close to the foci of targeted tumors. With the ability to describe the local light dose, PAT may help in treatment planning by guiding the positioning of optical fibers and adjusting the light delivered by each fiber. The optimized illumination may achieve maximum light delivery to target tissue while minimizing light delivery to background normal tissues and minimize unwanted and potentially therapeutic side effects. Besides treatment planning, SPAT may also help evaluate treatment efficacy by quantifying tissue hemodynamic changes during and after the PDT procedure. Finally, the design and operation of the system according to the present invention are compatible with existing ultrasound imaging and can greatly enhance the capability of conventional ultrasonography without affecting its original imaging functions.

Use of photoacoustic technology to monitor and guide PDT according to the present invention can be adapted to any situation where PDT is used in light of its high sensitivity and high specificity to tissue hemodynamic changes, and its ability to assess and optimize precise light delivery to treated tissues. Situations where photoacoustic technology can be used for monitoring and guiding PDT include, but are not limited to, PDT for treatment of prostate cancer, benign prostatic hypertrophy, tenosynovitis, nodular basal cell carcinoma, ampullary cancer, hepatocellular carcinoma, any superficial cancer including those of the skin, macular degeneration, bladder cancer, head and neck cancers, liver metastases, cholangiocarcinoma, skin rejuvenation, cutaneous skin and mucousal infections, endodontic infections, joint tissue destruction in rheumatic disease, penile intraepithelial neoplasia, CNS tumor ablation including gliomas, fibrosing dermopathies including scleroderma and nephrogenic fibrosing dermopathy, psoriasis, oral cancers, cutaneous lupus, and Barrett's esophagus.

Photoacoustic technology according to the present invention can also be adapted to the monitoring, guidance and evaluation of other therapeutic technologies beside PDT, for example radiation therapy and high intensity ultrasound therapy. Photoacoustic technology to monitor and guide PDT can be used in endoscopic settings including, but not limited to, colonoscopy, esophagogastroduodenoscopy, laparoscopy, rhinoscopy, sigmoidoscopy, laryngoscopy, bronchoscopy or nasopharyngoscopy, and in multi-modality systems incorporating other imaging and sensing technologies including, but not limited to, ultrasound, Doppler ultrasound, optical imaging and NIRS. Laser-generated ultrasound signals, or photoacoustic signals, can be sensed outside the body with external ultrasound sensors, e.g. ultrasonic transducers. Transducers with different geometries including, but not limited to, linear, arc, circular and 2D arrays can be applied according to the imaging requirement and the location of the imaged object. Photoacoustic signals produced by or not by PDT can also be measured inside any biologic substance including human or animal organs, tissues and vessels with more localized small ultrasonic transducers attached to, immediately next to, or at any distance from the light source.

Photoacoustic technology according to the present invention could also be utilized for sensing in the setting of photosensitized tagged or conjugated biologic substances such as human or animal molecular, cellular and tissue components. A specific example of this is incorporating photoacoustic technology into the setting of light-induced in situ patterning of DNA-tagged biomolecules and nanoparticles. Photoacoustic technology could also be utilized for sensing or altering in any way inherently, tagged or conjugated photosensitized non-biologic substances including, but not limited to, substances in either liquid, gas, or solid phase. One example of this includes tagging impurities in a liquid with a photosensitized substance followed by using localized laser light for destruction or alteration in any way of the same tagged impurities.

With reference to the system and method described herein, photoacoustic technologies present tissue structures and features, including those around optical sources, based on the intrinsic tissue optical contrast, which may help in finding the foci and borders of target tissues. Photoacoustic technologies describe light energy distribution and realize guided-light delivery during therapy, which may permit interactive adjustment to the direction and intensity of the light beam. In addition, photoacoustic technologies assess treatment efficacy by measuring local tissue blood oxygenation and blood volume before, during, and after therapy, which may permit interactive adjustment of treatment intensity for optimizing treatment outcome. Still further, photoacoustic technologies can be incorporated into multimodality imaging and sensing systems externally and in endoscopic settings with each modality in each setting being exploited for its imaging and sensing contribution in the setting of using PDT along with optical and ultrasound sources and transducers.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A system for monitoring photodynamic therapy of a target tissue, the target tissue containing a photosensitizing substance, the system comprising:
a first light source configured to deliver light to the target tissue, the first light source having a wavelength capable of exciting the photosensitizing substance;
an ultrasonic transducer for receiving photoacoustic signals generated due to optical absorption of light energy by the target tissue; and
a control unit in communication with the ultrasonic transducer for reconstructing photoacoustic tomographic images from the received photoacoustic signals to provide an indication of optical energy deposition due to the photosensitizing substance in the target tissue.

2. The system according to claim 1, wherein the first light source is configured to deliver short duration light pulses to the target tissue for imaging.

3. The system according to claim 2, wherein the first light source has a tunable wavelength.

4. The system according to claim 1, further comprising a second light source in communication with the control unit, the second light source configured to deliver short duration light pulses to the target tissue for imaging.

5. The system according to claim 4, wherein the first light source and the second light source operate at the same wavelength.

6. The system according to claim 4, wherein the first light source and the second light source operate at different wavelengths.

7. The system according to claim 4, wherein the second light source has a tunable wavelength.

8. The system according to claim 4, wherein the control unit receives a firing trigger from the second light source.

9. The system according to claim 4, wherein the control unit controls tuning the wavelength of the second light source.

10. The system according to claim 4, further comprising optical fibers which communicate light from the first light source and the second light source to the target tissue, wherein the optical fibers from each light source are joined by an optical coupler to deliver light from each light source to the same location in the target tissue.

11. The system according to claim 1, wherein upon delivery of light pulses of two or more wavelengths to the target tissue, the control unit is configured to determine the local spectroscopic absorption of the photosensitizing substance at any location in the target tissue.

12. The system according to claim 1, wherein upon delivery of light pulses of two or more wavelengths to the target tissue, the control unit is configured to determine an estimation of blood oxygenation in the target tissue.

13. The system according to claim 1, wherein the target tissue includes the prostate.

14. The system according to claim 1, wherein the control unit is in communication with the first light source for controlling the operation thereof.

15. The system according to claim 1, wherein the ultrasonic transducer is configured to transmit ultrasound signals to the target tissue for generating ultrasound images.

16. A system for monitoring photodynamic therapy of a target tissue, the target tissue containing a photosensitizing substance, the system comprising:
a first light source configured to deliver light to the target tissue, the first light source having a wavelength capable of exciting the photosensitizing substance;
a second light source configured to deliver short duration light pulses to the target tissue for imaging;
an ultrasonic transducer for receiving photoacoustic signals generated due to optical absorption of light energy by the target tissue; and
a control unit in communication with the ultrasonic transducer for reconstructing photoacoustic tomographic images from the received photoacoustic signals to provide an indication of optical energy deposition due to the photosensitizing substance in the target tissue and for determining an estimation of blood oxygenation in the target tissue.

17. A method for monitoring photodynamic therapy of a target tissue, the target tissue containing a photosensitizing substance, the method comprising:
providing a first light source for delivering light to the target tissue;
exciting the photosensitizing substance in the target tissue;
receiving photoacoustic signals generated due to optical absorption of light energy by the target tissue with an ultrasonic transducer; and
reconstructing photoacoustic tomographic images from the received photoacoustic signals to provide an indication of optical energy deposition of the photosensitizing substance in the target tissue.

18. The method according to claim 17, wherein the first light source is configured to deliver short duration light pulses to the target tissue for imaging.

19. The method according to claim 18, wherein the first light source has a tunable wavelength for delivering light pulses of two or more different wavelengths to the target tissue.

20. The method according to claim 17, further comprising providing a second light source configured to deliver short duration light pulses to the target tissue for imaging.

21. The method according to claim 20, wherein the second light source has a tunable wavelength for delivering light pulses of two or more different wavelengths to the target tissue.

21. The method according to claim 20, further comprising operating the first light source and the second light source at the same wavelength.

22. The method according to claim 20, further comprising operating the first light source and the second light source at different wavelengths.

23. The method according to claim 17, further comprising determining the local spectroscopic absorption of the photosensitizing substance at any location in the target tissue.

24. The method according to claim 17, further comprising determining an estimation of blood oxygenation in the target tissue.

25. The method according to claim 17, further comprising transmitting ultrasound signals to the target tissue for generating ultrasound images.

26. The method according to claim 17, further comprising scanning the ultrasonic transducer along an axis relative to the target tissue.
